(19) United States
(12) Patent Application Publication
(10) Pub. No.: US 2025/0135155 A1
(43) Pub. Date: May 1, 2025

(54) INDWELLING CATHETER

(71) Applicant: TERUMO KABUSHIKI KAISHA, Tokyo (JP)

(72) Inventors: Manabu MIURA, Hadano-shi (JP); Naoto TAKEMURA, Atsugi-shi (JP)

(73) Assignee: TERUMO KABUSHIKI KAISHA, Tokyo (JP)

(21) Appl. No.: 19/006,457

(22) Filed: Dec. 31, 2024

Related U.S. Application Data
(63) Continuation of application No. PCT/JP2023/024553, filed on Jul. 3, 2023.

(30) Foreign Application Priority Data
Jul. 7, 2022 (JP) 2022-109463

(51) Int. Cl. A61M 25/00

(52) U.S. Cl. CPC A61M 25/0045; A61M 2025/0046; A61M 2205/0216; A61M 2205/0222; A61M 2205/0238; A61M 2205/025

(57) ABSTRACT

An indwelling catheter configured to be indwelled in a living body lumen includes: a shaft including a lumen. An outer diameter of the shaft is 2.5 mm or less. An initial elasticity of the indwelling catheter in warm water at 37° C. is 0.012 N/mm to 0.13 N/mm. A shape restoring rate of the indwelling catheter in warm water at 37° C. is 73% or more. A sliding resistance of the indwelling catheter in physiological saline at 23° C. is 0.025 N or less.

### CROSS-REFERENCE TO RELATED APPLICATIONS

This application is a bypass continuation of PCT Application No. PCT/JP2023/024553, filed on Jul. 3, 2023, which claims priority to Japanese Patent Application No. 2022-109463, filed on Jul. 7, 2022. The entire contents of these applications are incorporated by references.

### BACKGROUND

The present disclosure relates to an indwelling catheter indwelled in a living body lumen such as a blood vessel to administer a predetermined administered substance.

Conventionally, there has been known an indwelling catheter indwelled in a living body lumen such as a blood vessel for administering an administered substance such as various nutrients and medical agents to an administration target (refer to JP 11-33107 A). In a procedure using a peripherally inserted central venous catheter (PICC), which is a type of indwelling catheters, for example, the catheter is introduced into the blood vessel from a puncture site formed in a vein (for example, basilic vein, cephalic vein, median cubital vein and the like) located in the periphery of a body such as a hand, an arm, or an elbow, and a distal end of the catheter is indwelled in a central vein (for example, superior vena cava) via an axillary vein or a subclavian vein.

### SUMMARY

In an indwelling catheter indwelled in a blood vessel, a catheter distal end position at the time of indwelling is important to appropriately administer an administered substance to a target site. If the distal end position is displaced, there is a case in which the administration cannot be appropriately performed, for example, the administration is performed toward another blood vessel, and there is a possibility that a sufficient effect by the administered substance cannot be expected.

Since the indwelling catheter is a device indwelled in the blood vessel to be used, a distal end might be displaced from the initial indwelling position due to body movement of a patient, recoil when a medical agent is administered and the like. For example, in a case in which the medical agent is administered via the catheter, the distal end is drawn toward a proximal end side due to the recoil when the medical agent is administered. In such a case, with a catheter having a high restoring property of the catheter distal end (distal end returning property), the distal end moved toward the proximal end side returns to the initial indwelling position again; however, with a catheter having a low restoring property, after being drawn from the initial indwelling position to the proximal end side, the catheter cannot return to the initial indwelling position and the distal end position remains displaced.

The inventors of the present application have intensively studied the properties of the catheter to enhance the distal end returning property of the catheter against such unintended positional displacement of the catheter distal end. The inventors of the present application have found that a mutual relationship between slipperiness of a surface of the catheter and physical properties of the catheter (shape restoring property (shape restoring rate) of the distal end shape and hardness (initial elasticity) of the catheter) is significantly involved in the distal end returning property of the catheter, and have developed the present invention.

At least one embodiment of the present disclosure has been made in view of the above-described circumstances, and specifically, an object of certain embodiments is to provide an indwelling catheter having an excellent distal end returning property for returning a distal end position to an initial indwelling position even when positional displacement occurs on a catheter distal end in a state of being indwelled in a living body lumen.

An indwelling catheter according to one embodiment includes a shaft including a lumen, the indwelling catheter indwelled in a living body lumen, in which an outer diameter is 2.5 mm or less, initial elasticity in warm water at 37° C. is 0.012 N/mm to 0.13 N/mm, a shape restoring rate in warm water at 37° C. is 73% or more, and sliding resistance in physiological saline at 23° C. is 0.025 N or less.

An indwelling catheter according to at least one embodiment of the present invention is provided with three characteristic physical properties of initial elasticity, a shape restoring rate, and sliding resistance, so that even if the position of the distal end is displaced from the initial indwelling position due to the positional displacement mechanism at the time of indwelling in the blood vessel, appropriate elasticity and slipperiness necessary for returning to the initial indwelling position are provided. Therefore, even if the distal end position is displaced in a state in which the indwelling catheter is indwelled in the living body lumen, this can return the distal end position to the initial indwelling position by exhibiting an excellent distal end returning property.

### BRIEF DESCRIPTION OF DRAWINGS

FIG. 1 is a schematic configuration diagram of an indwelling catheter according to one embodiment of the present invention;

FIG. 2 is a conceptual diagram for explaining positional displacement when a general indwelling catheter is indwelled in a blood vessel;

FIG. 3 is a schematic configuration diagram of a test device for measuring initial elasticity of the indwelling catheter;

FIG. 4 is a schematic configuration diagram of a test device for measuring a shape restoring rate of the indwelling catheter;

FIG. 5 is a schematic configuration diagram of a test device for measuring sliding resistance of the indwelling catheter;

FIG. 6 is a schematic configuration diagram of a test device for measuring a distal end returning rate of the indwelling catheter; and

FIG. 7 is a schematic configuration diagram of the test device illustrated in FIG. 6 as viewed from the side.

### DETAILED DESCRIPTION

Hereinafter, embodiments of the present disclosure will be described in detail with reference to the drawings. Embodiments described herein are illustrated to embody the technical ideas of the present invention, but the present invention is not limited to the described embodiments. Other feasible embodiments, examples, operation technologies and the like that could be conceived by those skilled in the art without departing from the gist of the present invention are all included in the scope and gist of the present invention and included in the invention recited in claims and the scope of equivalents thereof.

Moreover, for convenience of illustration and ease of understanding, the drawings attached to the present specification might be schematically represented by changing a scale, an aspect ratio, a shape and the like from actual ones as appropriate. The drawings are merely examples, and do not limit the interpretation of the present invention.

In the description of the present specification, a side on which an indwelling catheter 1 is inserted into a blood vessel is referred to as a “distal end side,” and a side opposite to the distal end side (a side gripped by an operator) is referred to as a “proximal end side.” A portion including a certain range in a long axis direction from a distal end (most distal end) is referred to as a “distal end portion,” and a portion including a certain range in the long axis direction from a proximal end (most proximal end) is referred to as a “proximal end portion.”

The indwelling catheter 1 according to the present embodiment is a medical device used for administering a predetermined administered substance to an administration target over a long period of time in a state of being indwelled in a living body lumen such as the blood vessel. The indwelling catheter 1 can be formed as a central venous catheter (CVC) for intravenous indwelling such as a PICC, for example. The indwelling catheter 1 configured as the central venous catheter is percutaneously inserted into the living body lumen (for example, the basilic vein, cephalic vein, median cubital vein and the like) by a Seldinger method and the like, and a distal end thereof is delivered to a target indwelling position (for example, the superior vena cava and the like) to be indwelled.

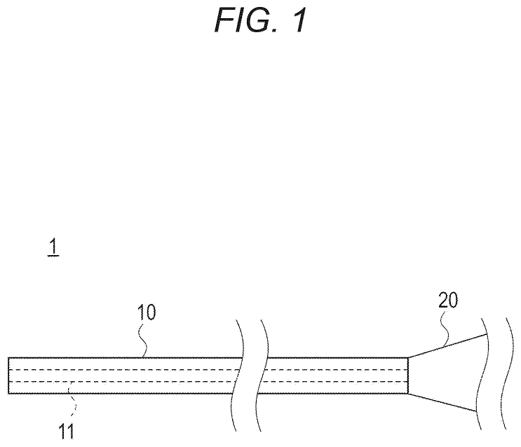

As illustrated in FIG. 1, the indwelling catheter 1 is a medical device including a lumen 11 through which the administered substance can circulate and formed of a long tubular member having flexibility that can be inserted into the blood vessel (vein).

A shaft 10 serving as a main body of the indwelling catheter 1 includes the lumen 11. A hub 20 serving as a port for administering the administered substance is attached to a proximal end side of the shaft 10. The number of lumens 11 of the shaft 10 is not limited to one, and may be plural. The hub 20 may include a plurality of ports in accordance with the number of lumens 11.

The shaft 10 may be formed of a material having flexibility. The material forming the shaft 10 is not particularly limited, but a material satisfying at least physical properties (initial elasticity and shape restoring property) of the indwelling catheter 1 to be described later is appropriately selected. The shaft 10 can be formed of, for example, polyolefins such as polyethylene, polypropylene, an ethylene-propylene copolymer, and an ethylene-vinyl acetate copolymer, thermoplastic resins such as soft polyvinyl chloride, various elastomers such as a polyurethane resin, a polyamide resin, and a polyester resin, crystalline plastics such as polyamide, crystalline polyethylene, and crystalline polypropylene, a silicon resin and the like. As the polyurethane resin, that containing a diisocyanate, a diol chain extender, and a polyglycol as main components, in which the diisocyanate is an aromatic diisocyanate, the diol is an aliphatic diol, and the polyglycol is an aromatic polyglycol or an aliphatic polyglycol can be suitably used. The shaft 10 can also use a mixture containing two or more kinds of the above-described materials as a constituent material.

Note that these materials may contain an antithrombotic material having antithrombogenicity and biocompatibility (effect of suppressing and preventing adhesion/adhesion of platelets, and effect of suppressing and preventing activation of platelets). The antithrombotic material includes a physiologically active substance having antithrombogenicity, an antithrombotic synthetic polymer material having a chemical structure exhibiting antithrombogenicity and suppressing adsorption of proteins and the like. Examples of the physiologically active substance include heparin, prostaglandin, urokinase, and arginine derivative, for example. Examples of the antithrombotic synthetic polymer material include a polymer material including zwitterionic monomers exhibiting antithrombogenicity, such as, for example, 2-methoxyethyl acrylate (2-MEA) and a betaine-type monomer (sulfobetaine-type monomer, carboxybetaine-type monomer such as phosphobetaine-type monomer containing a phosphorylcholine group such as 2-methacryloyloxyethyl phosphorylcholine (MPC)), for example. These antithrombotic materials may be used alone or in a form of a mixture of two or more of them.

In a case in which the indwelling catheter 1 is a device intended for being indwelled in the blood vessel (mainly vein) as the living body lumen, an outer diameter of the shaft 10 is 2.5 mm or less, preferably 1.0 mm or more and 1.7 mm or less.

An outer surface of the shaft 10 is provided with a lubricative coating that functions as a friction reducing portion for reducing sliding resistance at the time of insertion into the blood vessel and improving a distal end returning property for unintended displacement of a distal end position.

The lubricative coating is not particularly limited as long as this swells upon contact with a body fluid or an aqueous solvent and expresses high lubricity in a living body, and a material satisfying at least the physical property (sliding resistance) of the indwelling catheter 1 to be described later is appropriately selected. The lubricative coating can be formed of, for example, a low-friction material such as a hydrophilic material. Examples of the hydrophilic material include hydrophilic polymers such as cellulose-based polymer substances, polyethylene oxide-based polymer substances, maleic anhydride-based polymer substances (for example, a maleic anhydride copolymer such as a methyl vinyl ether-maleic anhydride copolymer), acrylamide-based polymer substances (for example, a block copolymer of polyacrylamide and glycidyl methacrylate-dimethylacrylamide), water-soluble nylon, polyvinyl alcohol, polyvinyl pyrrolidone, and derivatives thereof, for example.

The lubricative coating may contain an antithrombotic material (physiologically active substances, antithrombotic synthetic polymer materials and the like) having antithrombogenicity and biocompatibility. Note that, as the antithrombotic material contained in the lubricative coating, the antithrombotic material similar to that applicable as the constituent material of the shaft 10 described above can be used. As a result, the shaft 10 of the indwelling catheter 1 has excellent slipperiness, and can exhibit excellent antithrombogenicity even when this is indwelled in the blood vessel for a long period of time.

The outer surface of the indwelling catheter 1 may be coated with the lubricative coating using known coating treatment (such as dip coating or spray coating). The indwelling catheter 1 may have a configuration in which a functional layer having a friction reducing function during shaft molding is provided on the outer surface in advance, in addition to a configuration in which the outer surface is coated with a low-friction material by the coating treatment.

The administered substance administered from the indwelling catheter 1 can be appropriately selected according to a therapeutic purpose. The administered substance is a fluid composition that can express a predetermined effect by being administered to the living body as the administration target, and can be intermittently released from the device. The administered substance is, for example, a medical agent (liquid agent) intended for treatment of predetermined disease and various nutrients. The medical agent includes a drug. The drug can be any physiologically or pharmacologically active substance, in particular one known to be delivered to a human or animal body. Examples of the medical agent include, but are not limited to, an infusion agent, an anticancer agent, an antibacterial agent and the like, for example.

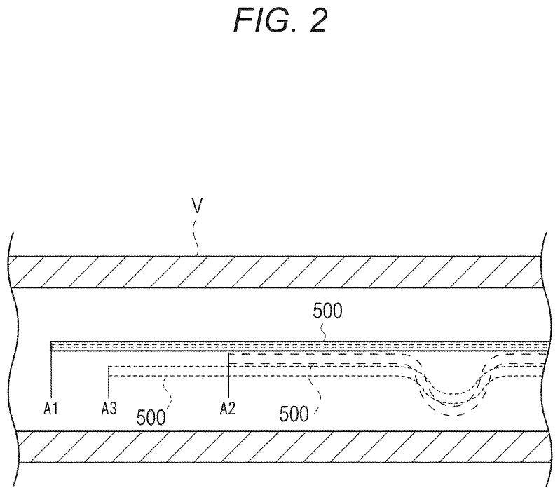

FIG. 2 is a diagram illustrating a state in which a general indwelling catheter 500 is indwelled in a blood vessel V (vein). When being introduced into the vein, the indwelling catheter 500 is indwelled with an indwelling position of a distal end in the blood vessel determined so that the administered substance is appropriately administered. For example, as illustrated in FIG. 2, the distal end of the indwelling catheter 500 is initially located at an indwelling position A1. However, in the indwelling catheter 500, the distal end might be drawn toward a proximal end side and drawn from the indwelling position A1 to be moved to a position A2 due to body movement of a patient or recoil at the time of administration of the administered substance. Thereafter, the distal end of the indwelling catheter 1 desirably returns to the position of the indwelling position A1 again, but might return only to a return position A3 in some cases as illustrated in FIG. 2. In a case in which the distal end position does not return to the initial indwelling position A1, there is a possibility that the administered substance is not administered to an appropriate site.

The indwelling catheter 1 according to the present embodiment is provided with the following physical properties for improving the distal end returning property to solve a problem caused by positional displacement of the distal end that might occur during indwelling in the blood vessel as illustrated in FIG. 2.

In the indwelling catheter 1, the outer diameter of the shaft 10 is set to 2.5 mm, “initial elasticity in warm water at 37° C. is set to 0.012 N/mm to 0.13 N/mm (first physical property),” “a shape restoring rate in warm water at 37° C. is set to 73% or more (second physical property),” and “sliding resistance in physiological saline at 23° C. is set to 0.025 N or less (third physical property).” Provided with these three characteristic physical properties, the indwelling catheter 1 is excellent in distal end returning property as compared with a known indwelling catheter, and can be returned to the initial indwelling position (or a position at which administration of the administered substance is not interfered) even if the positional displacement of the distal end occurs.

Hereinafter, the three physical properties (first to third physical properties) of the indwelling catheter 1 will be described in detail.

### <First Physical Property: Initial Elasticity>

The initial elasticity is a physical property value [N/mm] quantitatively indicating hardness of the shaft 10 of the indwelling catheter 1. The initial elasticity of the indwelling catheter 1 is uniquely defined by a type and a composition ratio of constituent materials, and a product size (inner and outer diameters and the like).

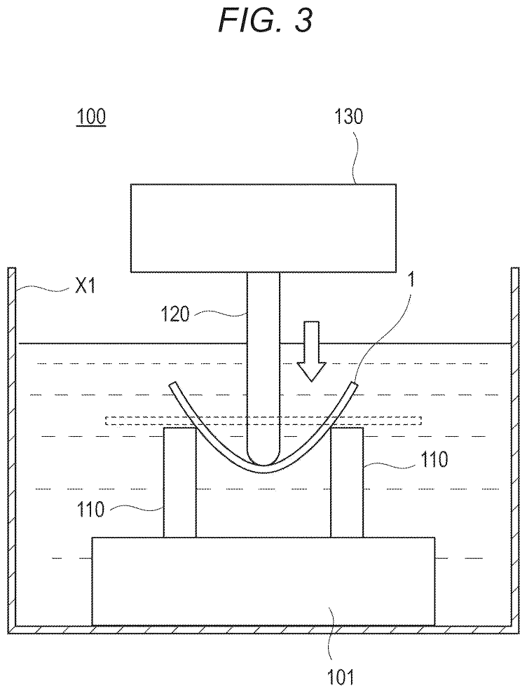

The initial elasticity can be measured using a test device 100 illustrated in FIG. 3. As illustrated in FIG. 3, the test device 100 is provided with a pair of supports 110 arranged on a base 101 at a predetermined interval (for example, 15 mm) in a long axis direction (extending direction) of the indwelling catheter 1 placed thereon, an indenter 120 that presses the indwelling catheter 1 placed on the supports 110 in a direction (vertical direction) orthogonal to the long axis direction, and a pressing drive unit 130 that adjusts a pressing load of the indenter 120. To reproduce a temperature environment in the living body, the test device 100 is performed in a water tank X1 storing warm water of temperature close to body temperature (for example, 37° C.).

As a measurement method using the test device 100, the indwelling catheter 1 is arranged on the supports 110, the pressing drive unit 130 is driven to move the indenter 120 so as to press the same by a predetermined pressing distance (for example, 0.1 mm) at a predetermined pressing speed (for example, 20 mm/min), and a pressing load [N] and the pressing distance [mm] are measured. The initial elasticity is a physical property value obtained on the basis of the pressing load [N] and the pressing distance [mm] when the indwelling catheter 1 is pressed by the indenter 120; the larger the value, the harder, and the smaller the value, the softer. The initial elasticity can also be said to be a proportional constant (inclination) at an initial stage of deformation between the pressing load and the pressing distance.

In the indwelling catheter 1 according to the present embodiment, in a case in which the distal end returning property for a positional displacement mechanism at the time of indwelling in the blood vessel is improved, the initial elasticity is preferably set in a range of 0.012 N/mm to 0.13 N/mm, and more preferably set in a range of 0.027 N/mm to 0.036 N/mm. By setting the initial elasticity of the indwelling catheter 1 within the above-described range, it is possible to exhibit an appropriate shape retaining property necessary for the distal end to return to the initial indwelling position when this is drawn toward the proximal end side, for example.

### <Second Physical Property: Shape Restoring Rate>

The shape restoring rate is a physical property value [%] quantitatively indicating a restoring property for the deformed shaft 10 of the indwelling catheter 1 to return to its original shape.

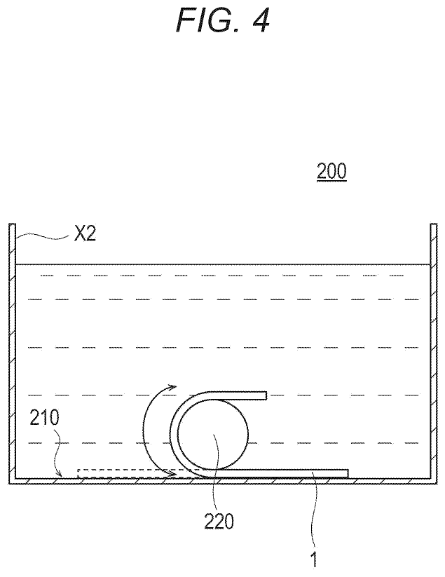

The shape restoring rate can be measured using a test device 200 illustrated in FIG. 4. As illustrated in FIG. 4, the test device 200 on a predetermined placing surface 210 (bottom surface of a water tank X2 in the drawing) of which the indwelling catheter 1 is placed is provided with a columnar support column 220 having a predetermined diameter (for example, φ7 mm) for winding the indwelling catheter 1 arranged away from the placing surface 210 by a predetermined distance. To reproduce a temperature environment in the living body, the test device 200 is performed in the water tank X2 storing warm water of temperature close to the body temperature (for example, 37° C.).

As a measurement method using the test device 200, the indwelling catheter 1 is arranged on the placing surface 210, the indwelling catheter 1 is wound around the column 220 by 180° and the indwelling catheter 1 is separated after a predetermined time elapses (for example, after one second) to release a wound state, and an angle between an outer surface facing the placing surface 210 and the placing surface 210 in the distal end portion of the indwelling catheter 1 after the winding is released is measured. The shape restoring rate is a physical property value on the basis of the angle between the indwelling catheter 1 and the placing surface 210 serving as a reference surface before and after winding of the indwelling catheter 1, and can be obtained from following Expression 1 by measuring the angle of the indwelling catheter 1 after the winding is released. The shape restoring rate indicates that the larger the value, the higher the restoring property, and the smaller the value, the lower the restoring property. For example, when the angle of the indwelling catheter 1 after the winding is released is 0°, the shape restoring rate is “100%,” which means the restoration to the original shape.

Expression 1: Angle from placing surface 210 after winding is released/180°×100%.

In the indwelling catheter 1 according to the present embodiment, in a case in which the distal end returning property for the positional displacement mechanism at the time of indwelling in the blood vessel is improved, the shape restoring rate is preferably set to 73% or more, and more preferably set to 79% or more. By setting the shape restoring rate within the above-described range, the indwelling catheter 1 can exhibit an appropriate shape restoring property necessary for the distal end to return to the initial indwelling position when this is drawn toward the proximal end side, for example.

### <Third Physical Property: Sliding Resistance>

The sliding resistance is a physical property value [N] quantitatively indicating ease of sliding when the indwelling catheter 1 is inserted into the blood vessel. That is, the sliding resistance means magnitude of resistance when moving the shaft 10 in a bent state in the axial direction relative to the lumen of a unit length.

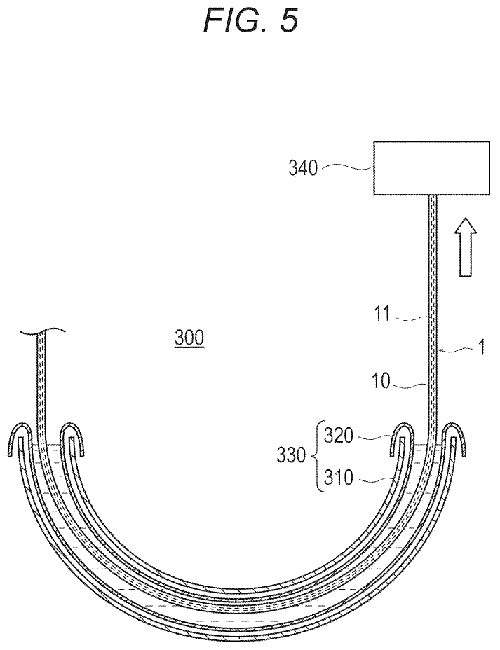

The sliding resistance can be measured using a test device 300 illustrated in FIG. 5. As illustrated in FIG. 5, the test device 300 is provided with a blood vessel model 330 formed of a resin tube 310 for reproducing elasticity of a blood vessel and a blood vessel (porcine jugular vein) 320 arranged along a lumen of the resin tube, and a tensile drive unit 340 for pulling the indwelling catheter 1 with a predetermined tensile force (for example, 5 mm/sec). The test device 300 is operated in a state in which the indwelling catheter 1 is inserted into the blood vessel model 330 filled with physiological saline (for example, 23° C.) in a temperature range from normal temperature to temperature close to body temperature.

As a measurement method using the test device 300, a tensile load [N] when the indwelling catheter 1 is inserted into the blood vessel model 330 and the tensile drive unit 340 is driven is measured. The sliding resistance is a physical property value indicating magnitude of the frictional resistance when passing through the blood vessel model 330, indicating that the larger the value, the higher the frictional resistance, and the smaller the value, the lower the frictional resistance.

In the indwelling catheter 1 according to the present embodiment, in a case in which the distal end returning property for the positional displacement mechanism at the time of indwelling in the blood vessel is improved, the sliding resistance is preferably set to 0.025 N or less. By setting the sliding resistance of the indwelling catheter 1 within the above-described range, it is possible to exhibit appropriate slipperiness necessary for the distal end to return to the initial indwelling position when this is drawn toward the proximal end side, for example.

As described above, the indwelling catheter 1 is provided with the three characteristic physical properties (initial elasticity, shape restoring rate, and sliding resistance) measured using the test devices 100, 200, and 300. The indwelling catheter 1 is a device excellent in distal end returning property. Therefore, even in a case in which the positional displacement of the distal end occurs in the indwelling catheter 1 in a state of being indwelled in the blood vessel, this can return to the initial indwelling position by exhibiting the excellent distal end returning property.

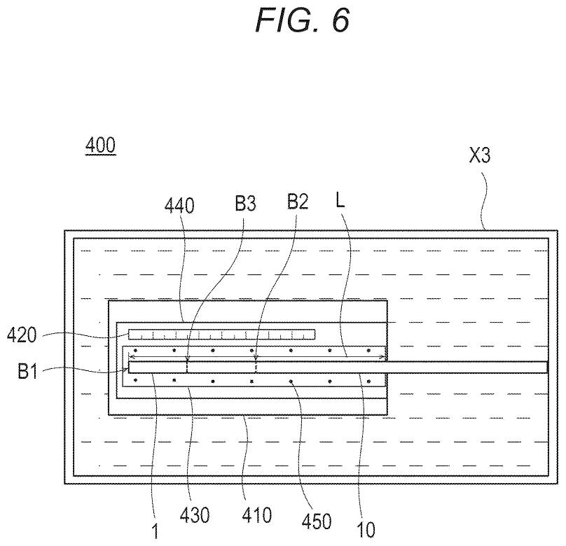

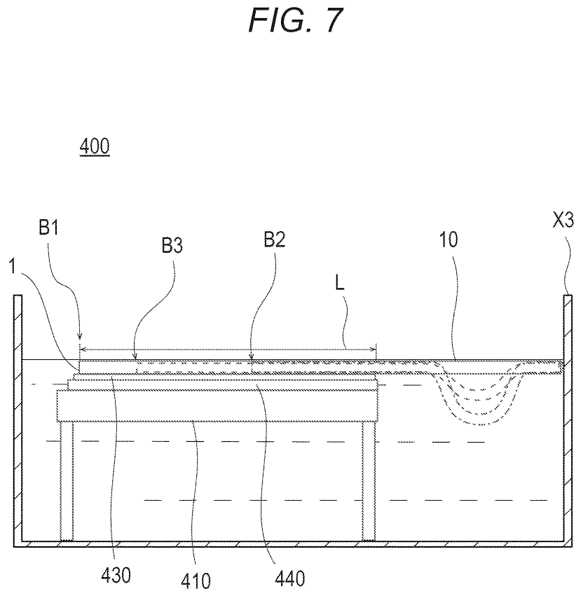

The distal end returning property of the indwelling catheter 1 can be evaluated by a distal end returning rate [%] obtained by a test device 400 illustrated in FIGS. 6 and 7. The distal end returning rate is a physical property value [%] for quantitatively evaluating how much it is possible to return to the initial indwelling position when the distal end position is displaced with reference to the initial indwelling position of the shaft 10 of the indwelling catheter 1.

As illustrated in FIGS. 6 and 7, the test device 400 is provided with a table 410 on which the indwelling catheter 1 is placed, a scale 420 placed on the table 410 for measuring a distance from an initial position B1 to a return position B3 of the indwelling catheter 1, a blood vessel 430 on which the indwelling catheter 1 is placed, a base material 440 made of silicone resin placed on the table 410 on which the blood vessel 430 is placed, and a fixing member 450 such as a gusset needle that fixes the blood vessel 430 onto the base material 440. The blood vessel 430 for which a porcine internal jugular vein is used, for example, is cut and incised in a long axis direction, and placed on the base material 440 in a state in which an inner wall of the blood vessel is exposed. The proximal end of the indwelling catheter 1 is fixed to an inner wall surface of a water tank X3. To reproduce a temperature environment in the living body, the test device 400 is performed in the water tank X3 storing warm water of temperature close to the body temperature (for example, 37° C.).

As a measurement method using the test device 400, the table 410 on which the scale 420 is arranged is installed in the water tank X3 storing warm water. The indwelling catheter 1 is placed on the blood vessel 430 in parallel with the scale 420, and a distal end position is aligned to a scale of zero of the scale 420. To accurately measure the initial position B1 and the return position B3 when placing the indwelling catheter 1 on the blood vessel 430, a contact distance L (for example, 90 mm) between the blood vessel 430 and the shaft 10 in a state in which the indwelling catheter 1 is placed on the blood vessel 430 is defined in advance. To suppress the movement of the indwelling catheter 1 due to a water flow that might occur during the test, a height of a water surface of the warm water stored in the water tank X3 is set substantially equivalent to a height of an uppermost surface of the indwelling catheter 1. Next, after the distal end of the indwelling catheter 1 is drawn from the initial position B1 toward the proximal end side by a predetermined distance (for example, 25 mm) and moved to a drawn position B2, the drawing is released. A drawing/releasing operation the distal end position of the indwelling catheter 1 can be performed, for example, by pressing the proximal end side of the indwelling catheter 1 in a bottom direction of the water tank X3 with a finger of an examiner and the like and then releasing the pressing. In FIGS. 6 and 7, the shaft 10 of the indwelling catheter 1 is indicated by a solid line in a state in which the distal end position is the initial position B1, by a dashed-dotted line in a state in which the distal end position is the drawn position B2, and by a dotted line in a state in which the distal end position is the return position B3. The distal end returning rate is a physical property value based on the distance from the initial position B1 of the distal end of the indwelling catheter 1 to the return position B3 after the pressing is released, the larger the value, the higher the distal end returning property, and the smaller the value, the lower the distal end returning property. Therefore, 100% is obtained when the return position B3 of the distal end returns to the initial position B1.

The indwelling catheter 1 according to the present embodiment is provided with the three characteristic physical properties (initial elasticity, shape restoring rate, and sliding resistance), the distal end returning rate is 96% or more. Therefore, even in a case in which the positional displacement of the distal end occurs in the indwelling catheter 1 in a state of being indwelled in the blood vessel, this can return to the initial indwelling position (or a position at which administration of the administered substance is not interfered) by exhibiting the excellent distal end returning property.

### [Operational Effects]

As described above, the indwelling catheter according to the present embodiment is a catheter provided with the shaft 10 including the lumen 11 and is indwelled in the living body lumen, the catheter having the outer diameter of 2.5 mm or less, the initial elasticity in warm water at 37° C. of 0.012 N/mm to 0.13 N/mm, the shape restoring rate in warm water at 37° C. of 73% or more, and the sliding resistance in physiological saline at 23° C. of 0.025 N or less. The indwelling catheter 1 preferably has the initial elasticity of 0.027 N/mm to 0.036 N/mm, and the shape restoring rate of 79% or more.

The indwelling catheter 1 is provided with the three characteristic physical properties of the initial elasticity, the shape restoring rate, and the sliding resistance in the above-described range, so that even if the position of the distal end is displaced from the initial indwelling position due to the positional displacement mechanism at the time of indwelling in the blood vessel, this is provided with appropriate elasticity and slipperiness necessary for returning to the initial indwelling position. Therefore, even if the distal end position is displaced in a state in which the indwelling catheter 1 is indwelled in the living body lumen, this can return the distal end position to the initial indwelling position by exhibiting the excellent distal end returning property.

In the indwelling catheter 1 according to the present embodiment, it is preferable that the lubricative coating is applied to the outer surface of the shaft 10, and further, it is particularly preferable that the lubricative coating is a physiologically active substance having antithrombogenicity or a configuration having a chemical structure exhibiting antithrombogenicity (a configuration containing an antithrombotic material such as a physiologically active substance having antithrombogenicity and an antithrombotic synthetic polymer material having a chemical structure exhibiting antithrombogenicity and suppressing adsorption of proteins).

With such a configuration, the indwelling catheter 1 has a configuration having appropriate slipperiness contributing to improvement in distal end returning property, and can smoothly return to a target indwelling position without damaging the inner wall of the blood vessel at the time of a return operation of the distal end. When the lubricative coating contains an antithrombotic material, generation of thrombus due to indwelling of the shaft 10 is suppressed, and long-term indwelling in a blood vessel becomes possible.

In the indwelling catheter 1 according to the present embodiment, the shaft 10 may contain a polyurethane resin or may contain a silicone resin.

With such configuration, it is possible to manufacture the indwelling catheter 1 having the initial elasticity as the first physical property and the shape restoring rate as the second physical property necessary for improving the distal end returning property.

### Examples

Hereinafter, while the present invention will be specifically described with reference to Examples, the scope of the present invention is not limited to the following Examples.

Examples and Comparative Examples of the indwelling catheter according to the embodiment of the present invention will be described.

### [Test Outline]

For the indwelling catheters prepared in Examples and Comparative Examples, “initial elasticity,” “shape restoring property,” “sliding resistance,” and “distal end returning rate” were measured as physical properties according to the following test conditions and measurement procedures.

### <Initial Elasticity Test>

An initial elasticity test is a test in which the initial elasticity of each sample is measured using a test device 100 illustrated in FIG. 3. As the measurement procedure of the initial elasticity test, first, the test device 100 illustrated in FIG. 3 was prepared. The test device 100 had a configuration in which a pair of supports 110 were arranged on a base 101 at an interval of 15 mm. The test device 100 was placed in a water tank X1 storing warm water at 37° C. Next, the sample was placed on the supports 110, and the initial elasticity [N/mm] of each sample was obtained on the basis of a pressing load [N] and a pressing distance [mm] when an indenter 120 was driven according to driving conditions (pressing speed: 20 mm/min, pressing distance: 0.1 mm).

### <Shape Restoring Rate Test>

A shape restoring rate test is a test in which a shape restoring rate of each sample is measured using a test device illustrated in FIG. 4. As a measurement procedure of the shape restoring rate test, first, a test device 200 illustrated in FIG. 4 was prepared. The test device 200 had a configuration in which a bottom surface of a water tank X2 serves as a placing surface 210 on which the sample is placed, and is provided with a columnar (φ7 mm) support column 220 around which the sample is wound. The test device 200 was indwelled in the water tank X2 storing warm water at 37° C. Next, the sample was placed on the placing surface 210 and wound around the support column 220 by 180°, and the shape restoring rate [%] was obtained on the basis of an angle between an outer surface facing the placing surface 210 and the placing surface 210 in a distal end portion of each sample when the winding state was released after one second. The shape restoring rate was calculated by Expression 1 (angle from placing surface 210 after winding was released/180°×100%).

### <Sliding Resistance Test>

A sliding resistance test is a test in which sliding resistance of each sample is measured using a test device 300 illustrated in FIG. 5. As a measurement procedure of the sliding resistance test, first, the test device 300 illustrated in FIG. 5 was prepared. The test device 300 had a configuration including a blood vessel model 330 in which a lumen of a polypropylene tube 310 (inner diameter of 4.5 mm, total length of 105 mm, degree of curvature (R): diameter of 6.5 cm) is covered with a blood vessel 320 (porcine jugular vein (inner diameter of 2.5 mm to 4 mm, total length of 100 mm)), and a tensile drive unit 340 that pulls the sample. Next, the blood vessel model 330 was filled with physiological saline at 23° C., the sample was inserted into the blood vessel model 330, and then the sliding resistance [N] of each sample was obtained on the basis of the tensile load [N] when the sample was pulled out at a drawing speed of 5 mm/sec.

### <Distal End Returning Rate Test>

A distal end returning rate test is a test in which a returning rate before and after movement of a distal end portion of each sample is measured using a test device 400 illustrated in FIGS. 6 and 7. As a measurement procedure of the distal end returning rate test, first, the test device 400 illustrated in FIG. 6 was prepared. In the test device 400, a table 410 was installed in a water tank X3 storing warm water at 37° C., and each sample and a scale 420 were placed in parallel on the table 410. A height of a water surface of warm water stored in the water tank X3 was substantially equivalent to a height of an uppermost surface of each sample. The scale 420 was arranged with a scale of zero aligned to an initial position B1 in a long axis direction of the sample in such a manner that a moving distance of the sample on the proximal end side can be grasped. A base material 440 made of silicone resin was placed on the table 410, and the blood vessel 430 for placing the sample was placed thereon. The blood vessel 430 for which a porcine internal jugular vein is used is cut and incised in a long axis direction, and placed in a state in which an inner wall of the blood vessel is exposed. The blood vessel 430 was fixed to the base material 440 by piercing a plurality of fixing members (gusset needles) 450 along both side portions so as not to move during the test. When the sample was placed on the blood vessel 430, a contact distance L between the blood vessel 430 and each sample was 90 mm. A proximal end of each sample was fixed to an inner wall surface of the water tank X3. Next, as illustrated in FIG. 7, the proximal end side of the sample was pressed in a bottom direction of the water tank X3 (depth direction in the drawing) with a finger of an examiner so that the distal end of the sample moved from the initial position B1 toward the proximal end side to a position of 25 mm, and then the pressing was released, and a distal end returning property [%] of each sample was obtained on the basis of the distance from the initial position B1 to the position where the distal end of the sample returned (return position B3). Note that, in FIGS. 6 and 7, the shaft 10 of the indwelling catheter 1 is indicated by a solid line in a state in which the distal end position is the initial position B1, by a dashed-dotted line in a state in which the distal end position is the drawn position B2, and by a dotted line in a state in which the distal end position is the return position B3.

### [Sample Specifications]

Table 1 illustrates specifications of each sample used in Examples and Comparative Examples.

TABLE 1

| | | | DIMENSION | |
|---|---|---|---|---|
| | | LUBRICATIVE | OUTER | INNER |
| | MATERIAL | COATING | DIAMETER | DIAMETER |
| EXAMPLE 1 | POLYCARBONATE-BASED | YES | 1.14 | 0.75 |
| | POLYURETHANE | | | |
| EXAMPLE 2 | POLYETHER-BASED | YES | 1.08 | 0.70 |
| | POLYURETHANE (PTMG, MDI) | | | |
| EXAMPLE 3 | POLYETHER-BASED | YES | 0.96 | 0.64 |
| | POLYURETHANE (PTMG, | | | |
| | HYDROGENATED MDI) | | | |
| EXAMPLE 4 | POLYETHER-BASED | YES | 1.40 | 0.84 |
| | POLYURETHANE | | | |
| EXAMPLE 5 | SILICONE | YES | 0.99 | 0.63 |
| EXAMPLE 6 | POLYCAPROLACTONE-BASED | YES | 0.83 | 0.62 |
| | POLYURETHANE | | | |
| EXAMPLE 7 | POLYCAPROLACTONE-BASED | YES | 0.85 | 0.62 |
| | POLYURETHANE | | | |
| COMPARATIVE | POLYCARBONATE-BASED | NO | 1.14 | 0.75 |
| EXAMPLE 1 | POLYURETHANE | | | |
| COMPARATIVE | POLYETHER-BASED | NO | 1.08 | 0.70 |
| EXAMPLE 2 | POLYURETHANE (PTMG, MDI) | | | |
| COMPARATIVE | POLYETHER-BASED | NO | 0.96 | 0.64 |
| EXAMPLE 3 | POLYURETHANE (PTMG, | | | |
| | HYDROGENATED MDI) | | | |
| COMPARATIVE | POLYETHER-BASED | NO | 1.40 | 0.84 |
| EXAMPLE 4 | POLYURETHANE | | | |
| COMPARATIVE | SILICONE | NO | 0.99 | 0.63 |
| EXAMPLE 5 | | | | |
| COMPARATIVE | POLYCAPROLACTONE-BASED | NO | 0.83 | 0.62 |
| EXAMPLE 6 | POLYURETHANE | | | |
| COMPARATIVE | POLYCAPROLACTONE-BASED | NO | 0.85 | 0.62 |
| EXAMPLE 7 | POLYURETHANE | | | |
| COMPARATIVE | SILICONE | YES | 0.70 | 0.30 |
| EXAMPLE 8 | | | | |
| COMPARATIVE | POLYURETHANE, STAINLESS | YES | 1.14 | 0.75 |
| EXAMPLE 9 | COPPER | | | |
| COMPARATIVE | POLYURETHANE, COPPER | YES | 0.83 | 0.62 |

| | |
|---|---|
| EXAMPLE 10 | WIRE |

### [Results]

A measurement results of Examples and Comparative Examples are illustrated in Table 2.

TABLE 2

| | INITIAL | SHAPE | SLIDING | DISTAL END |
|---|---|---|---|---|
| | ELASTICITY | RESTORING | RESISTANCE | RETURNING |
| | [N/mm] | RATE [%] | [N] | RATE [%] |
| EXAMPLE 1 | 0.079 | 83 | 0.018 | 98.1 |
| EXAMPLE 2 | 0.036 | 93 | 0.021 | 100.0 |
| EXAMPLE 3 | 0.012 | 89 | 0.020 | 96.3 |
| EXAMPLE 4 | 0.026 | 81 | 0.025 | 100.0 |
| EXAMPLE 5 | 0.031 | 100 | 0.023 | 100.0 |
| EXAMPLE 6 | 0.027 | 79 | 0.009 | 100.0 |
| EXAMPLE 7 | 0.014 | 73 | 0.016 | 96.0 |
| COMPARATIVE | 0.079 | 83 | 0.038 | 93.8 |
| EXAMPLE 1 | | | | |
| COMPARATIVE | 0.036 | 93 | 0.028 | 94.0 |
| EXAMPLE 2 | | | | |
| COMPARATIVE | 0.012 | 89 | 0.027 | 16.0 |
| EXAMPLE 3 | | | | |
| COMPARATIVE | 0.026 | 81 | 0.029 | 93.8 |
| EXAMPLE 4 | | | | |
| COMPARATIVE | 0.031 | 100 | 0.060 | 32.0 |
| EXAMPLE 5 | | | | |
| COMPARATIVE | 0.027 | 79 | 0.023 | 86.0 |
| EXAMPLE 6 | | | | |
| COMPARATIVE | 0.014 | 73 | 0.025 | 50.0 |
| EXAMPLE 7 | | | | |
| COMPARATIVE | 0.002 | 100 | 0.019 | 0.0 |
| EXAMPLE 8 | | | | |
| COMPARATIVE | 0.131 | 97 | 0.041 | CANNOT BE |
| EXAMPLE 9 | | | | MEASURED |
| COMPARATIVE | 0.048 | 50 | 0.015 | 75.0 |
| EXAMPLE 10 | | | | |

As illustrated in Table 2, in Examples 1 to 7, the distal end returning rate exceeded 96%. This indicates that, by setting the initial elasticity, which is a physical property of the indwelling catheter, to a range of 0.012 N/mm to 0.13 N/mm, setting the shape restoring rate to 73% or more, and setting the sliding resistance to 0.025 N or less, these physical properties function effectively to enhance the distal end returning property of the indwelling catheter. In particular, in Example 2 and Examples 4 to 6, the initial elasticity was in the range of 0.027 N/mm to 0.036 N/mm, and the shape restoring rate was 79% or more, so that the distal end returning rate was 100%, indicating that more excellent distal end returning properties than those of Examples 1, 3, and 7 can be obtained.

In contrast, as illustrated in Table 2, in Comparative Examples 1 to 10, the distal end returning rate was lower than 96%. This is presumed to be due to the fact that all the physical property values of the initial elasticity, shape restoring rate, and sliding resistance of each Comparative Example do not satisfy the ranges of Examples.

As described above, to improve the distal end returning property in the indwelling catheter, it has been proved that it is effective to set the initial elasticity to the range of 0.012 N/mm to 0.13 N/mm, set the shape restoring rate to 73% or more, set the sliding resistance to 0.025 N or less, and more preferably set the initial elasticity to the range of 0.027 N/mm to 0.036 N/mm, and set the shape restoring rate to 79% or more.

What is claimed is:

1. An indwelling catheter configured to be indwelled in a living body lumen, the indwelling catheter comprising:
a shaft including a lumen, wherein:
an outer diameter of the shaft is 2.5 mm or less,
an initial elasticity of the indwelling catheter in warm water at 37° C. is 0.012 N/mm to 0.13 N/mm,
a shape restoring rate of the indwelling catheter in warm water at 37° C. is 73% or more, and
a sliding resistance of the indwelling catheter in physiological saline at 23° C. is 0.025 N or less.

2. The indwelling catheter according to claim 1, wherein
the initial elasticity is 0.027 N/mm to 0.036 N/mm, and the shape restoring rate is 79% or more.

3. The indwelling catheter according to claim 1, further comprising a lubricative coating disposed on an outer surface of the shaft.

4. The indwelling catheter according to claim 2, further comprising a lubricative coating disposed on an outer surface of the shaft.

5. The indwelling catheter according to claim 3, wherein the lubricative coating includes a physiologically active substance having antithrombogenicity or has a chemical structure exhibiting antithrombogenicity.

6. The indwelling catheter according to claim 4, wherein the lubricative coating includes a physiologically active substance having antithrombogenicity or has a chemical structure exhibiting antithrombogenicity.

7. The indwelling catheter according to claim 1, wherein the shaft contains a polyurethane resin.

8. The indwelling catheter according to claim 2, wherein the shaft contains a polyurethane resin.

9. The indwelling catheter according to claim 1, wherein the shaft contains a silicon resin.

10. The indwelling catheter according to claim 2, wherein the shaft contains a silicon resin.

11. A method comprising:
inserting an indwelling catheter in a living body lumen, the indwelling catheter comprising:
a shaft including a lumen, wherein:
an outer diameter of the shaft is 2.5 mm or less,
an initial elasticity of the indwelling catheter in warm water at 37° C. is 0.012 N/mm to 0.13 N/mm,
a shape restoring rate of the indwelling catheter in warm water at 37° C. is 73% or more, and
a sliding resistance of the indwelling catheter in physiological saline at 23° C. is 0.025 N or less.

12. The method according to claim 11, wherein
the initial elasticity is 0.027 N/mm to 0.036 N/mm, and the shape restoring rate is 79% or more.

13. The method according to claim 11, further comprising a lubricative coating disposed on an outer surface of the shaft.

14. The method according to claim 12, further comprising a lubricative coating disposed on an outer surface of the shaft.

15. The method according to claim 13, wherein the lubricative coating includes a physiologically active substance having antithrombogenicity or has a chemical structure exhibiting antithrombogenicity.

16. The method according to claim 14, wherein the lubricative coating includes a physiologically active substance having antithrombogenicity or has a chemical structure exhibiting antithrombogenicity.

17. The method according to claim 11, wherein the shaft contains a polyurethane resin.

18. The method according to claim 12, wherein the shaft contains a polyurethane resin.

19. The method according to claim 11, wherein the shaft contains a silicon resin.

20. The method according to claim 1, wherein the living body lumen is a vein.
